(19) United States
(12) Patent Application Publication
(10) Pub. No.: US 2017/0252390 A1
(43) Pub. Date: Sep. 7, 2017

(54) TOPICAL SKIN CARE FORMULATION

(71) Applicant: Regina Monibi, Reno, NV (US)

(72) Inventor: Regina Monibi, Reno, NV (US)

(21) Appl. No.: 15/518,185

(22) PCT Filed: Oct. 6, 2015
(86) PCT No.: PCT/US15/54243

§ 371 Date: Apr. 10, 2017

Related U.S. Application Data
(60) Provisional application No. 62/062,564, filed on Oct. 10, 2014.

(51) Int. Cl. A61K 36/48; A61K 47/18; A61K 9/00; A61K 8/97; A61K 8/41; A61Q 19/08; A61K 8/92; A61K 8/67; A61K 8/49; A61K 8/365; A61K 8/64; A61K 8/44; A61K 36/28; A61K 8/73

(52) U.S. Cl. CPC A61K 36/48; A61K 36/28; A61K 47/18; A61K 9/0014; A61K 8/97; A61K 8/41; A61K 8/735; A61K 8/922; A61K 8/975; A61K 8/678; A61K 8/676; A61K 8/671; A61K 8/4986; A61K 8/365; A61K 8/64; A61K 8/44; A61Q 19/08

(57) ABSTRACT

The present disclosure is drawn to a topical skin care formulation comprising Calendula and Rooibos. Other ingredients can optionally be included, such as DMAE.

### BACKGROUND

The cosmetic industry covers a wide variety of products which can include naturally or synthetically derived compounds for application to the skin. Compounds are often formulated into skin care compositions which are applied to the skin for: cleansing, moisturizing, deodorizing, enhancing or altering ones appearance, etc. Cosmetics come in a variety of forms including, but not limited to, creams, lotions, powders, oils, gels, salts, butters, emollients, tinctures, and serums, to name a few. Specific formulations that include organic and/or naturally derived cosmetic compounds for reducing and/or diminishing the appearance of age-related dermatological symptoms and various other skin conditions continue to be sought after.

### SUMMARY

A topical skin care formulation can comprise from 0.25 wt % to 30 wt % Calendula, and from 0.25 wt % to 30 wt % Rooibos. In one embodiment, the topical skin care formulation can comprise from 0.25 wt % to 15 wt % Calendula, and from 0.25 wt % to 15 wt % Rooibos. In one specific example, the formulation can also include dimethylethanolamine (DMAE).

In another embodiment, a method of treating a dermatological condition can comprise applying a topical skin care formulation to a skin surface. The topical skin care formulation can comprise from 0.25 wt % to 30 wt % Calendula, and from 0.25 wt % to 30 wt % Rooibos. In one specific example, the formulation can also include dimethylethanolamine (DMAE).

### DETAILED DESCRIPTION

In the present disclosure, the following terminology will be used in accordance with the definitions set forth below.

The singular forms “a,” “an,” and “the” include plural referents unless the context clearly dictates otherwise. Thus, for example, reference to “a serum” includes reference to one or more serum.

In this disclosure, “comprises,” “comprising,” “containing” and “having” and the like can have the meaning ascribed to them in U.S. patent law and can mean “includes,” “including,” and the like, and are generally interpreted to be open ended terms. The term “consisting of” is a closed term, and includes only the devices, methods, compositions, components, structures, steps, or the like specifically listed, and that which is in accordance with U.S. patent law. “Consisting essentially of” or “consists essentially” or the like, when applied to devices, methods, compositions, components, structures, steps, or the like encompassed by the present disclosure, refers to elements like those disclosed herein, but which may contain additional structural groups, composition components, method steps, etc. Such additional structural groups, composition components, methods steps, etc., however, do not materially affect their basic and novel characteristic(s) compared to those of the corresponding structural groups, composition components, methods steps, etc. disclosed herein. When using an open ended term, like “comprising” or “including,” it is understood that direct support should be afforded also to “consisting essentially of” language as well as “consisting of” language as if stated explicitly.

As used herein, a plurality of compositional compounds and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range includes “about ‘x’ to about ‘y’.” To illustrate, a concentration range of “about 0.1 wt % to about 5 wt %” should be interpreted to include not only the explicitly recited concentration of about 0.1 wt % to about 5 wt %, but also to include individual concentrations (e.g., 1 wt %, 2 wt %, 3 wt %, and 4 wt %) and the sub-ranges (e.g., 0.5 wt %, 1.1 wt %, 2.2 wt %, 3.3 wt %, and 4.4 wt %) within the indicated range. In one embodiment, the term “about” can include traditional rounding according to significant figures of the numerical value. In addition, the phrase “about ‘x’ to ‘y’” includes “about ‘x’ to about ‘y’.”

The phrase, “age-related dermatological symptoms or conditions” refers to visible symptoms on the surface of skin that occurs as a consequence of aging. These symptoms include, but are not limited to include: wrinkles, fine lines, creases, age spots, blemishes, dry skin, or the like.

“Skin” is defined as human skin.

“Calendula” refers to compounds from the plant genus Calendula, commonly referred to as marigold, and is often in the form of an extract. In one example, the Calendula that is used excludes extracts containing tagetes.

“Rooibos” refers to compounds from the plant Aspalathus lineris, commonly referred to as the red bush. Rooibos is often included in the formulations of the present disclosure as an extract.

The term “extract” includes substances prepared by extracting raw materials using solvent, e.g., ethanol or water, using supercritical CO₂, or by other known extraction methods. These extracts typically have a given purity percentage, and can be relatively to highly pure. However, it is noted that when describing the wt % of Rooibos, Calendula, or other ingredients of the formulations of the present disclosure, the wt % of an ingredient is based on its pure form. Thus, alcohols, water, other solvents, or other co-dispersed compounds in solid or liquid form that may be present residually as part of an extract can be subtracted out for purposes of determining the wt % of a specific ingredient in the formulation as a whole. The same would be true for ingredients added that are not in the form of extracts.

When referring to achieving an effect, phrases such as “reduction in appearance of,” “reducing the appearance of,” or “diminishes the appearance of,” one or more condition(s) refers to a visible reduction in the condition(s) that are noticeable to the naked eye. This reduction can occur over a period of time. The appearance of the reduction is counted from the subject's appearance on day 1 to the subject's appearance some period of time after a given period of time of consistent use, e.g., 1 week, 2 weeks, 1 month, 3 months, 1 year, etc. The degree of visible reduction can vary based on a variety of factors, including but not limited to, the skin type of the subject, the skin condition that is being improved, and/or the individual characteristics of the subject.

As will be apparent to those of skill in the art upon reading this disclosure, each of the individual embodiments described and illustrated herein has discrete components and features that may be readily separated from or combined with the features of any of the other several embodiments without departing from the scope or spirit of the present disclosure. Any recited method can be carried out in the order of events recited or in any other order that is logically possible.

With this in mind, a topical skin care formulation can comprise a combination of Calendula and Rooibos. In certain embodiments, the Calendula and Rooibos can each be present at from about 0.25 wt % to about 30 wt %. In another embodiment, the Calendula and Rooibos can each be present from about 0.25 wt % to about 15 wt % of the formulation. In other embodiments, the Calendula and Rooibos can each be present at from about 0.25 wt % to about 10 wt % of the formulation. In other embodiments, the Calendula can be present at from about 1 wt % to about 10 wt % of the formulation. In still other embodiments the Rooibos can be present at about 1 wt % to 5 wt % of the formulation.

In further detail regarding the ingredients of Calendula and Rooibos, Calendula contains polyphenolic substances that are a good source of natural antioxidants. The polyphenolic substances in Calendula are suspected to provide the beneficial effects to the skin when applied in accordance with examples of the present disclosure. Calendula can be added in the form of Calendula extract, or can be added as part of an ingredient in another cream or similar formulation.

Rooibos (Aspalathus linearis) grows primarily in South Africa. The ingredient can be added as an extract or from other creams or similar formulations that include Rooibos. Rooibos extract, for example, has been formulated and has been used in herbal teas. The health benefits of Rooibos may be attributable to its high antioxidant level and the potency of the antioxidants.

Calendula and Rooibos when combined together in a common skin care formulation can provide a potent and unusually effective formulation for the treatment of age-related dermatological conditions. The formulation disclosed herein which comprises a combination of Calendula and Rooibos, among other ingredients, has been found to be effective at reducing and diminishing the appearance of age-related dermatological conditions, as well as acne, stretch marks, cellulite, scars, eczema, and psoriasis.

As briefly mentioned, Calendula and Rooibos can be added to the formulations of the present disclosure as a pure or relatively pure extract, in a cream, in a butter, in a butter cream, in a serum, in a tincture, or any combination thereof. The formulations are intended for topical application, and thus, can be applied using any form suitable for topical application. Non-limiting examples of forms suitable for topical application include, but are not limited to include: serums, creams, emulsions, tinctures, emollients, lotions, oils, butters, cleansers, masks, patches, gels, or the like.

In certain embodiments, the formulation can comprise additional ingredients, including at least one of: dimethylethanolamine (DMAE), alpha lipoic acid, hyaluronic acid, alpha hydroxy acid, vitamin A, vitamin C, vitamin E, algae extract, acetylhexapeptide-30 and arginine solution (commercially available as Inyline™ solution), aloe butter, aloe jelly, argan oil, cocoa butter, lemon grass essential oil, rose hip butter, shea butter, orange blossom water, other ingredients typically found in cosmetic formulations, and combinations thereof. The additional ingredients can be added in any form that can be formulated in a topical application. For example, when the additional ingredient DMAE is added, the DMAE may be added in the form of a serum, jelly, or combination thereof. To provide some more specific examples, the formulation may include Calendula, Rooibos, and DMAE. If DMAE is added, in one example, the DMAE can be included at from 0.25 wt % to 10 wt %, or more typically from 0.25 wt % to 5 wt %.

The additional ingredients can be used to further enhance the beneficial properties of the formulation or as a body for the formulation. DMAE can be used, for example, to firm, smooth, and brighten skin. Alpha lipoic acid can be used as an antioxidant to reduce the appearance of fine lines. Hyaluronic acid can be used topically to moisturize the skin. Alpha hydroxy acid can be used to reduce the appearance of wrinkles, the signs of aging, and improve the look and feel of the skin. Vitamin A can be used cosmetically to reduce the appearance of skin conditions including: acne, eczema, psoriasis, sunburn, and pigmentation. Vitamin A can be used to reduce the appearance of wrinkles. Vitamin C can be used to protect the skin, reduce the appearance of wrinkles, and reduce acne. Vitamin E can be used as an antioxidant that can protect the skin from ultra-violet radiation. Algae extract can be used in the cosmetic industry to reduce skin problems associated with excess oil. Inyline™ solution (which includes water, acetyl hexapeptide-30, arginine, and caprylyl glycol) can also be used to reduce the appearance of wrinkles by smoothing the skin surface and decreasing line depth. A variety of aloes, butters, and creams can likewise be used as a base cream, and/or to moisturize the skin and provide body to cosmetic formulations.

The topical skin care formulation can be applied to reduce and diminish the appearance of age-related dermatological conditions and various other skin conditions. These conditions include: fine lines, creases, wrinkles, blemishes, age spots, acne, stretch marks, cellulite, scars, eczema, and psoriasis. The formulation can also be used to protect human skin from ultraviolet radiation, and/or to stimulate an increase in collagen production.

The present disclosure also further sets forth a method for using the formulation. In one embodiment, this method can include topically applying the formulation to a skin surface of a subject once a day, twice a day, or three times a day. In another embodiment, the method can include topically applying the formulation to the skin surface once a week, twice a week, three times a week, every other day, or daily. This process can be carried out indefinitely, or until the desired result is achieved. For example, the treatment may be carried out for 2 weeks, 1 month, 3 months, or 1 year.

The skin care treatment formulation is intended to be used in several embodiments as a skin care system. A combination of a Calendula and Rooibos can be formulated in the form of a: cleanser, a facial mask, a facial scrub, a serum, a cream, and a serum cream. The method of using the formulation can thus include multiple steps: (1) cleansing the face and neck with the cleanser every morning and/or night, (2) using either a mask or the scrub containing Calendula and Rooibos (and optionally DMAE) at least twice a week after cleansing, (3) applying a serum containing Calendula and Rooibos (and optionally DMAE) at least twice a day either after cleansing or, when using the mask or scrub, after using the mask the scrub, and/or (4) liberally applying a cream containing Calendula and Rooibos (and optionally DMAE) after the serum twice a day and throughout the day as needed. Optionally, this regimen can be modified for the needs of a particular individual. For examples, an older user with more wrinkles may use the product more often, or may use a product with a higher concentration of Rooibos and/or Calendula.

### EXAMPLES

While the following examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims.

### Example 1

A topical skin care formulation is prepared having the following ingredients as set forth in Table 1, as follows:

TABLE 1

| | |
|---|---|
| Calendula | 7.43 wt % |
| DMAE | 0.93 wt % |
| Rooibos | 0.93 wt % |
| Vitamin C | 0.24 wt % |

Additional formulation ingredients can include butters and/or creams used as a base, as well as other ingredients commonly found in face creams and moisturizers.

### Example 2

A topical skin care formulation is prepared having the following ingredients as set forth in Table 2, as follows:

TABLE 2

| | |
|---|---|
| Rooibos | 4.95 wt % |
| DMAE | 2.48 wt % |
| Calendula | 2.36 wt % |
| Hyaluronic Acid | 1.57 wt % |

Additional formulation ingredients can include butters and/or creams used as a base, as well as other ingredients commonly found in face creams and moisturizers.

### Example 3

A topical skin care formulation is prepared having the following ingredients as set forth in Table 3, as follows:

TABLE 3

| | |
|---|---|
| Calendula | 8.39 wt % |
| Rooibos | 1.05 wt % |
| DMAE | 0.52 wt % |
| Vitamin C | 0.27 wt % |

Additional formulation ingredients can include butters and/or creams used as a base, as well as other ingredients commonly found in face creams and moisturizers.

### Example 4

A topical skin care formulation is prepared having the following ingredients as set forth in Table 4, as follows:

TABLE 4

| | |
|---|---|
| DMAE | 2.28 wt % |
| Rooibos | 2.28 wt % |
| Calendula | 1.87 wt % |
| Hyaluronic Acid | 0.73 wt % |

This formulation further included aloe jelly, argan oil, vitamin C, vitamin E and orange blossom water. The formulation can further include butters, creams, and/or other ingredients commonly found in face creams and moisturizers.

### Example 5

A topical skin care formulation is prepared, as set forth in Table 5, as follows:

TABLE 5

| | |
|---|---|
| Orange Blossom Water | 2.398 wt % |
| DMAE Serum | 4.715 wt % |
| DMAE Jelly | 1.199 wt % |
| Rooibos Serum | 4.715 wt % |
| Paramount Facial Moisturizer | 37.721 wt % |
| Vitamin C Serum | 2.398 wt % |
| Calendula Butter Cream | 37.721 wt % |
| Shea Butter | 0.639 wt % |
| Vitamin E Butter | 2.398 wt % |
| Vitamin A Butter | 0.639 wt % |
| Rose Hip Butter | 0.639 wt % |
| Aloe Butter | 1.199 wt % |
| Cocoa Butter | 1.199 wt % |
| Argan Oil | 1.199 wt % |
| Lemon Grass Essential Oil | 0.039 wt % |
| Alpha Lipoic Acid | 0.394 wt % |
| Calendula Tincture | 0.394 wt % |
| Rooibos Tincture | 0.394 wt % |

### Example 6

A topical skin care formulation is prepared, as set forth in Table 6, as follows:

TABLE 6

| | |
|---|---|
| DMAE Serum | 4.973 wt % |
| DMAE Jelly | 1.264 wt % |
| Rooibos Serum | 4.973 wt % |
| Paramount Facial Moisturizer | 39.786 wt % |
| Vitamin C Serum | 2.529 wt % |
| Calendula Butter Cream | 39.786 wt % |
| Shea Butter | 0.674 wt % |
| Vitamin E Butter | 2.529 wt % |
| Vitamin A Butter | 0.674 wt % |
| Rose Hip Butter | 0.674 wt % |
| Argan Oil | 1.264 wt % |
| Lemon Grass Essential Oil | 0.042 wt % |
| Alpha Lipoic Acid | 0.416 wt % |
| Rooibos Tincture | 0.416 wt % |

### Example 7

A topical skin care formulation is prepared, as set forth in Table 7, as follows:

TABLE 7

| | |
|---|---|
| Paramount Facial Cream | 38.736 wt % |
| Calendula Butter Cream | 38.736 wt % |
| Rooibos Serum | 4.842 wt % |
| DMAE Serum | 4.842 wt % |
| Vitamin C Serum | 2.462 wt % |
| DMAE Jelly | 1.231 wt % |
| Rose Hip Butter | 0.657 wt % |
| Aloe Butter | 1.231 wt % |
| Shea Butter | 0.657 wt % |
| Vitamin A | 0.657 wt % |
| Vitamin E | 2.462 wt % |
| Rooibos Tincture | 0.164 wt % |
| Calendula Tincture | 0.164 wt % |
| Hyaluronic Acid | 0.410 wt % |
| Alpha Lipoic Acid | 0.082 wt % |
| Alpha Hydroxy Acid | 0.082 wt % |
| Algae Extract | 0.041 wt % |
| Inyline ™ Solution | 0.041 wt % |
| Orange Blossom Water | 2.462 wt % |
| Lemon Grass Essential Oil | 0.041 wt % |

### Example 8

A topical skin care formulation is prepared, as set forth in Table 8, as follows:

TABLE 8

| | |
|---|---|
| Paramount Facial Cream | 41.863 wt % |
| Calendula Butter Cream | 41.863 wt % |
| Rooibos Serum | 5.232 wt % |
| DMAE Jelly | 1.330 wt % |
| Rose Hip Butter | 0.710 wt % |
| Aloe Butter | 1.330 wt % |
| Shea Butter | 0.710 wt % |
| Vitamin A | 0.710 wt % |
| Vitamin E | 2.661 wt % |
| Calendula Tincture | 0.177 wt % |
| Hyaluronic Acid | 0.443 wt % |
| Alpha Lipoic Acid | 0.089 wt % |
| Alpha Hydroxy Acid | 0.089 wt % |
| Algae Extract | 0.044 wt % |
| Inyline ™ Solution | 0.044 wt % |
| Orange Blossom Water | 2.661 wt % |
| Lemon Grass Essential Oil | 0.044 wt % |

### Example 9

A topical skin care formulation is prepared, as set forth in Table 9, as follows:

TABLE 9

| | |
|---|---|
| Paramount Facial Cream | 39.105 wt % |
| Calendula Butter Cream | 39.105 wt % |
| Rooibos Serum | 4.888 wt % |
| DMAE Serum | 4.888 wt % |
| Vitamin C Serum | 2.486 wt % |
| DMAE Jelly | 1.243 wt % |
| Aloe Butter | 1.243 wt % |
| Shea Butter | 0.663 wt % |
| Vitamin A | 0.663 wt % |
| Vitamin E | 2.486 wt % |
| Rooibos Tincture | 0.166 wt % |
| Hyaluronic Acid | 0.414 wt % |
| Alpha Hydroxy Acid | 0.082 wt % |
| Inyline ™ Solution | 0.041 wt % |
| Orange Blossom Water | 2.486 wt % |
| Lemon Grass Essential Oil | 0.041 wt % |

### Example 10

A topical skin care formulation is prepared, as set forth in Table 10, as follows:

TABLE 10

| | |
|---|---|
| DMAE | 0.46 wt % |
| Rooibos | 3.60 wt % |
| Calendula | 4.01 wt % |

This formulation further comprises vitamin E, argan oil, and alpha lipoic acid, and may also include other butters and ingredients commonly found in face creams and moisturizers.

### Example 11

A topical skin care formulation is prepared, as set forth in Table 11, as follows:

TABLE 11

| | |
|---|---|
| Rooibos | 27.00 wt % |
| Calendula | 3.00 wt % |

Additional formulation ingredients can include butters and/or creams used as a base, as well as other ingredients commonly found in face creams and moisturizers. This formulation can also optionally include DMAE.

### Example 12

A topical skin care formulation is prepared, as set forth in Table 12, as follows:

TABLE 12

| | |
|---|---|
| Rooibos | 5.00 wt % |
| Calendula | 25.00 wt % |

Additional formulation ingredients can include butters and/or creams used as a base, as well as other ingredients commonly found in face creams and moisturizers. This formulation can also optionally include DMAE.

### Example 13

A topical skin care formulation is prepared which includes algae extract, aloe jelly, argan oil, Calendula butter cream, Calendula tincture, cocoa butter, DMAE serum, hyaluronic acid, orange blossom water, Rooibos serum, and vitamin E butter. The formulation was calculated to include approximately: 2.86 wt % DMAE, 2.86 wt % Rooibos, and 1.49 wt % Calendula.

As a note, ingredients found in Examples 1-13 can be acquired from Essential Wholesale & Labs located in Portland, Oreg. Inyline™ solution is available from Lotioncrafter, LLC located in Eastsound, Wash.

### Example 14

A 46 year old woman with dry skin applied the formulation prepared in accordance with Example 2 twice daily after cleaning her face. In addition, the woman applied the formulation in Example 1 every morning and night. The individual reported: a softening in the appearance of her wrinkles, the disappearance of fine lines, that her skin felt moisturized throughout the entire day, that she had a healthier skin tone, that her skin felt clean, and that she no longer experienced acne break outs.

### Example 15

A female applied the formulation in Example 1 twice a day to her hands, feet, and body. Within a few weeks, the individual noticed a decrease in wrinkles, a diminished appearance of a surgical scar, a diminished appearance of age spots, and a minimization in the amount of cellulite on her legs.

### Example 16

A female applied the formulation in Example 1 twice daily to eczema patches on her face. The appearance of the eczema patches diminished within a period of a few weeks.

### Example 17

A male with severe acne applied the formulation in Example 1 twice daily to his face. The appearance of the acne was diminished over a period of a few weeks.

While the disclosure has been described with reference to certain examples, those skilled in the art will appreciate that various modifications, changes, omissions, and substitutions can be made without departing from the spirit of the disclosure. It is intended, therefore, that the present disclosure be limited only by the scope of the following claims.

What is claimed is:

1. A topical skin care formulation, comprising:
from 0.25 wt % to 30 wt % Calendula, and
from 0.25 wt % to 30 wt % Rooibos.

2. The topical skin care formulation of claim 1, wherein the formulation comprises:
from 0.25 wt % to 15 wt % Calendula, and
from 0.25 wt % to 15 wt % Rooibos.

3. The topical skin care formulation of claim 1, wherein the formulation is formulated as a cleanser, facial mask, facial scrub, serum, cream, butter, serum cream, or a combination thereof.

4. The topical skin care formulation of claim 1, wherein the Calendula is provided by a source selected from the group consisting of Calendula butter cream, Calendula cream, Calendula butter, Calendula tincture, Calendula extract, and a combination thereof.

5. The topical skin care formulation of claim 1, wherein the Calendula is present in the formulation at from about 1 wt % to about 10 wt %.

6. The topical skin care formulation of claim 1, wherein the Rooibos is provided by a source selected from the group consisting of Rooibos serum, Rooibos tincture, Rooibos cream, Rooibos extract, and a combination thereof.

7. The topical skin care formulation of claim 1, wherein the Rooibos is present in the formulation at from about 1 wt % to about 5 wt %.

8. The topical skin care formulation of claim 1, wherein the Calendula and the Rooibos are each present at from about 0.25 wt % to about 10 wt %.

9. The topical skin care formulation of claim 1, further comprising dimethylaminoethanol (DMAE).

10. The topical skin care formulation of claim 9, wherein the DMAE is provided by a source selected from the group consisting of DMAE serum, DMAE jelly, and a combination thereof.

11. The topical skin care formulation of claim 9, wherein the DMAE is present at from 0.25 wt % to 10 wt %.

12. The topical skin care formulation of claim 1, further comprising at least one vitamin selected from the group consisting of alpha lipoic acid, hyaluronic acid, alpha hydroxy acid, vitamin A, vitamin C, Vitamin E, and a combination thereof.

13. The topical skin care formulation of claim 1, further comprising DMAE, hyaluronic acid, and orange blossom water.

14. The topical skin care formulation of claim 13, further comprising cocoa butter, argan oil, aloe jelly, vitamin E butter, and algae extract.

15. The topical skin care formulation of claim 13, further comprising aloe jelly, argan oil, vitamin E, and vitamin C.

16. The topical skin care formulation of claim 13, further comprising rose hip butter, aloe butter, shea butter, vitamin A, vitamin E, alpha lipoic acid, alpha hydroxy acid, algae extract, acetylhexapeptide-30, arginine, and lemon grass essential oil.

17. The topical skin care formulation of claim 13, further comprising vitamin A, vitamin C, vitamin E, aloe butter, shea butter, alpha hydroxy acid, acetylhexapeptide-30, arginine, and lemon grass essential oil.

18. The topical skin care formulation of claim 17, further comprising rose hip butter, alpha lipoic acid, and algae extract.

19. The topical skin care formulation of claim 1, further comprising DMAE, vitamin C, and argan oil.

20. The topical skin care formulation of claim 19, further comprising shea butter, vitamin A, vitamin E, rose hip butter, alpha lipoic acid, and lemon grass essential oil.

21. The topical skin care formulation of claim 20, further comprising aloe butter, cocoa butter, and orange blossom water.

22. The topical skin care formulation of claim 1, further comprising DMAE, hyaluronic acid, alpha lipoic acid, and vitamin E.

23. The topical skin care formulation of claim 1, further comprising: DMAE, vitamin C, acetylhexapeptide-30, arginine, and algae extract.

24. A method of treating dermatological condition, comprising applying a topical skin care formulation to a skin surface in need of treatment for the dermatological condition, said topical skin care formulation comprising from 0.25 wt % to 30 wt % Calendula and from 0.25 wt % to 30 wt % Rooibos.

25. The method of claim 24, wherein the formulation is formulated as a cleanser, facial mask, facial scrub, serum, cream, butter, serum cream, or a combination thereof.

26. The method of claim 24, wherein the Calendula and the Rooibos are each present at from about 0.25 wt % to about 10 wt %.

27. The method of claim 24, wherein the formulation further comprises dimethylaminoethanol (DMAE).

28. The method of claim 24, wherein the dermatological condition includes an age-related dermatological symptom selected from group consisting of wrinkles, fine lines, creases, and age spots.

29. The method of claim 24, wherein the dermatological condition includes a skin condition selected from the group consisting of photo-damage from ultraviolet radiation exposure, psoriasis, eczema, scars, cellulite, stretch marks, and acne.

30. The method of claim 24, wherein the formulation is applied at least twice a day for at least two weeks.
